(19) United States
(12) Patent Application Publication
(10) Pub. No.: US 2009/0220557 A1
(43) Pub. Date: Sep. 3, 2009

(54) EFFECT PIGMENT AND USE THEREOF IN COSMETICS AND IN THE FOOD AND PHARMACEUTICAL INDUSTRIES

(76) Inventors: Gerhard Pfaff, Muenster (DE); Johann Dietz, Dietzenbach (DE); Cornelia Foerderer, Heppenheim (DE); Xenia Petsitis, Hofheim am Taunus (DE); Doreen Warthe, Pfungstadt (DE); Veronika Hochstein, Bruchsal (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/094,080

(22) PCT Filed: Nov. 7, 2006
(86) PCT No.: PCT/EP2006/010669

§ 371 Date: May 16, 2008

(30) Foreign Application Priority Data
Nov. 18, 2005 (DE) 10 2005 055 576.4

(51) Int. Cl. A61K 8/02; A61K 9/14; A61K 33/26; B01J 20/02; A01N 25/26; C09K 3/00; C09K 15/00; F21V 9/06; C04B 14/04; A23L 1/27; C09B 67/08

(52) U.S. Cl. 424/401; 106/457; 427/218; 424/489; 424/646; 502/407; 424/421; 252/380; 252/397; 424/65; 424/59; 252/588; 106/401; 426/540; 8/623; 8/632

(57) ABSTRACT

The present invention relates to intensely coloured and light-stable effect pigments based on transparent, undoped SiO₂ flakes, which are distinguished by the fact that they are covered with an iron-oxide layer having a thickness of 30-150 nm, and all SiO₂ flakes have an identical thickness, and the thickness of the SiO₂ flakes is 250-400 nm, and to the use thereof in pigment mixtures, in cosmetic formulations and for colouring foods and pharmaceutical products.

The present invention relates to light-stable effect pigments based on thin transparent SiO₂ flakes covered with an iron-oxide layer, and to the use thereof in pure form or mixed with other colorants and/or fillers in cosmetic formulations and in the foods and pharmaceuticals sector.

WO 93/08237 discloses effect pigments based on transparent silicon dioxide flakes. The pigments described therein consist of thin SiO₂ flakes (thickness of the flakes from 50 μm to 5000 μm) which are covered with a thin metal-oxide layer or a plurality of metal-oxide layers. Instead of metal oxides, the SiO₂ flakes may also be covered with other materials, such as metals, sulfides or nitrides.

WO 20041044060 A1, WO 2004/065492 A1 and EP 1556446 A1 disclose, for example, metal oxide-coated SiO₂ flakes, where the parameter z can adopt the following values:

0.7≦z≦2.0, 0.03≦z≦2.0 or 0.7≦z≦1.8.

The metal oxide-coated SiO₂ flakes on the market with the trade name Xirona® from Merck KGaA consist of SiO₂ flakes of uniform thickness (±5 nm) covered with a metal-oxide layer (TiO₂ or Fe₂O₃) having a thickness of 10 nm to 500 nm. The thicknesses of the SiO₂ flakes are in the range from 200 nm to 900 nm. SiO₂ pigments of this type are distinguished, depending on the thickness of the flakes employed and the metal-oxide layers applied and on the nature of the metal oxide, by particularly intense interference colours and/or by strong angle-dependent colour-change effects. The latter are evident in such a way that an observer perceives different colours on changing his observation position to the pigmented object.

Intensely coloured red effect pigments based on SiO₂ flakes are described in DE 102005002124. These pigments comprise iron oxide-coated SiO₂ flakes, where the total thickness of the pigments is not greater than 500 nm (±30 nm).

Red effect pigments based on SiO₂ flakes are distinguished by their high lustre, their colour purity and tinting strength and should therefore be interesting, in particular, for cosmetics and for colouring food and pharmaceutical products if they are light-stable, do not bleed/migrate, promote a soft skin feel, are non-toxic and have high hiding power.

However, pigments based on SiO₂ flakes have the disadvantage that they do not have high hiding power owing to their high transparency. The object of the present invention was to provide a red effect pigment based on SiO₂ flakes which is not only light-stable, but also has comparatively high hiding power, can be incorporated very well into the respective application system and at the same time only influences the optical properties, such as, in particular, the lustre and colour purity, to an insignificant extent or not at all. They are chemically very stable both in the aqueous and oily phase, i.e. they do not bleed. They can therefore be employed both for the eye and also for the lip area.

Surprisingly, effect pigments have now been found which, besides an extraordinarily high tinting strength, have high light stability. These pigments are distinguished by the fact that they are based on transparent, undoped SiO₂ flakes which are covered with an iron-oxide layer having a thickness of 30-150 nm, preferably 60±30 nm, where all SiO₂ flakes have an identical thickness, and the thickness of the SiO₂ flakes is 250-400 nm, preferably 270 to 370 nm. Pigments of this type do not exhibit any colour changes in the Suntest and are thus very light-stable.

The hiding power of the pigments according to the invention is significantly increased if these pigments are mixed in combination with organic and inorganic fillers and/or with flake-form, needle-shaped, spherical or crystalline colorants. The admixing of one or more colorants with these effect pigments enables colour effects to be enhanced and novel colour effects to be achieved. Furthermore, the pigment mixtures are distinguished by their high lustre and a very good skin feel.

The invention thus relates to intensely coloured and light-stable effect pigments based on transparent, undoped SiO₂ flakes, characterised in that they are covered with an iron-oxide layer having a thickness of 30-150 nm, and all SiO₂ flakes have an identical thickness, and the thickness of the SiO₂ flakes is 250-400 nm.

The invention furthermore relates to a pigment mixture consisting of at least two components A and B, where component A comprises the intensely coloured and light-stable effect pigments according to Claim 1 and component B comprises inorganic pigments, organic pigments, dyes and/or fillers which consist of flake-form, needle-shaped, spherical or irregularly shaped particles.

The outstanding colouristic parameter is the strong, pure red colour of the pigments together with an optimum lustre effect. The iron oxide here is in the form of Fe₂O₃. The red effect pigments have Lab values in the range from L=20 to 200; a=20 to 100; b=10 to 70 (ETA measurement system, angle-dependent) and are therefore highly suitable for cosmetics, in particular decorative cosmetics.

The invention likewise relates to cosmetic formulations, such as, for example, make-ups, compact powders, loose powders, lipsticks, lotions, emulsions, etc., which comprise the pigment mixture according to the invention. The pigment mixtures are furthermore suitable for colourings and coloured coatings and for the finishing of foods and pharmaceutical products, including OTC preparations, such as, for example, medicament coatings of tablets, dragees, gelatin capsules, etc.

The red effect pigments can be mixed with the colorant or filler in any weight ratio. The ratio of component A to component B is preferably 99:1 to 50:50, in particular 95:5 to 70:30, very particularly preferably 70:30 to 50:50.

Preferred red effect pigments have the following structure:
SiO₂ flakes having a thickness of 250-400 nm, preferably 300-370 nm, in particular 340-365 nm+
Fe₂O₃ layer having a thickness of 60-250 nm, preferably 60-150 nm, in particular 80-120 nm.

Particular preference is given to red effect pigments in which the thicknesses of the SiO₂ flakes and of the iron-oxide layer are selected in such a way that the total thickness of the red effect pigments is not greater than 500 (±30) nm, preferably ≦440 (±30) nm, in particular <400 (±30) nm.

In order to achieve specific colour effects, finely divided particles in the nanometre size range may additionally be introduced into the Fe₂O₃ layer. Suitable for this purpose have proven to be, for example, finely divided TiO₂ or finely divided carbon (carbon black) having particle sizes in the range 10-250 nm. The light-scattering properties of such particles enable the lustre and hiding power to be influenced specifically.

The SiO₂ flakes can be produced, for example, as described in WO 93/08237.

The SiO₂ flakes can be coated with iron-oxide layers by wet-chemical methods and/or by CVD methods. Before application of the iron-oxide layer, a thin dielectric layer where 1.4<n<2.7 can optionally also be deposited. A coating of this type on SiO₂ flakes can consist, for example, of an Al₂O₃, ZnO or TiO₂ layer with a thickness of 2-20 nm and a colouristically matched Fe₂O₃ layer on top.

Preference is given to the use of the wet-chemical method for the preparation of the red effect pigments, where use can be made of the known wet-chemical coating technologies which were developed for the preparation of pearlescent pigments and are described, for example, in the following publications: DE 14 67 468, DE 19 59 988, DE 20 09 566, DE 22 14 545, DE 22 15 191, DE 22 44 2985 DE 23 13 331, DE 25 22 572, DE 31 37 808, DE 31 37 809, DE 31 51 343, DE 31 51 354, DE 31 51 355, DE 32 11 602, DE 32 35 017.

For the coating, the SiO₂ flakes are suspended in water and coated completely, i.e. covered, with iron oxide by addition and precipitation of corresponding inorganic iron compounds, where the pH necessary for the precipitation of iron oxide, preferably Fe₂O₃, is set and kept constant by simultaneous addition of acid or base, and the coated substrate is subsequently separated off from the aqueous suspension, dried and optionally calcined, and where the layer thicknesses of the individual layers are set in such a way that the thickness of the pigment after drying and optionally calcination is not greater than 500 (±30) nm, preferably <440 (±30) nm. The calcination temperature is generally between 250 and 1000° C., preferably between 350 and 900° C.

In principle, CVD methods for the coating of particles with iron oxide are also suitable for the production of the pigments according to the invention. Methods of this type are described, for example, in W. Ostertag, Nachr. Chem. Tech. Lab 1994, 42, 849. It is necessary here for the substrate to be kept uniformly in motion during the coating operation in order that a homogeneous coating of all particle surfaces is ensured and the substrate is covered completely and no open edges remain.

The red effect pigments can also be provided with an organic or inorganic protective layer in order to improve the light, weather and chemical stability or in order to increase the compatibility in various media. Suitable post-coatings or post-treatments are, for example, silanes, silicones, adsorbent silicones, metal soaps, amino acids, lecithins, fluorine components, polyethylenes, collagen or the methods described in DE 22 15 191, DE 31 51 354, DE 32 35 017 or DE 33 34 598, EP 0 632 109, U.S. Pat. No. 5,759,255, DE 43 17 019, DE 39 29 423, EP 0 492 223, EP 0 342 533, EP 0 268 918, EP 0 141 174, EP 0 764 191, WO 98/13426 or EP 0 465 805. This post-coating further increases the chemical and photochemical stability or simplifies handling of the red pigment, in particular incorporation into various media. In order to improve the wettability, dispersibility and/or compatibility with the user media, it is possible to apply, for example, functional coatings of Al₂O₃ or ZrO₂ or mixtures thereof to the pigment surface. Organic post-coatings, for example with silanes, as described, for example, in EP 0090259, EP 0 634 459, WO 99/57204, WO 96/32446, WO 99/57204, U.S. Pat. No. 5,759,255, U.S. Pat. No. 5,571,851, WO 01/92425 or in J. J. Ponjeé, Philips Technical Review, Vol. 44, No. 3, 81 ff. and P. H. Harding J. C. Berg, J. Adhesion Sci. Technol. Vol. 11 No. 4, pp. 471-493, are furthermore possible. The additionally applied substances make up only about 0.1 to 5% by weight, preferably 0.5 to 3.0% by weight, of the entire pigment.

The post-coating of the red effect pigments can be carried out directly in a one-pot process onto the Fe₂O₃ coating of the SiO₂ flakes. However, it is also possible firstly to isolate, optionally dry and calcine the red effect pigment and subsequently to apply the post-coating.

Besides the red effect pigment (component A), pigment mixtures according to the invention comprise a filler and/or a colorant of component B.

Suitable as component B for the pigment mixture according to the invention are all flake-form, needle-shaped, spherical and crystalline colorants or fillers which are known to the person skilled in the art, in particular those which have a particle size of 0.001 to 10 μm, preferably 0.01 to 1 μm. The pigment mixtures according to the invention preferably comprise absorption pigments as colorants and flake-form or spherical powders as fillers. Component B preferably comprises coated or uncoated SiO₂ beads. SiO₂ beads coated with one or more metal oxides are disclosed, for example, in EP 0 803 550 A2.

Besides component A, preferred pigment mixtures comprise in particular a pearlescent pigment, including multilayered pigments or interference pigments, as colorant (component B). The pearlescent pigments used are pigments based on flake-form, transparent or semitransparent substrates comprising, for example, phyllosilicates, such as, for example, natural or synthetic mica, talc, sericite, kaolin or other silicate materials, coated with coloured or colourless metal oxides, such as, for example, TiO₂, titanium suboxides, titanium oxynitrides, Fe₂O₃, Fe₃O₄, FeOOH, SnO₂, Cr₂O₃, ZnO, Quo, NiO, and other metal oxides, alone or in a mixture, in a single layer or in successive layers. Particularly preferred pearlescent pigments and interference pigments are based on a synthetic or natural mica flake (synthetic or natural mica), glass, Al₂O₃ or SiO₂ flake, furthermore Fe₂O₃ flake which is coated with titanium dioxide (rutile or anatase), Fe₂O₃, a mixture of TiO₂ and Fe₂O₃ or with Fe₃O₄.

Pearlescent pigments are disclosed, for example, in German patents and patent applications 14 67 468, 19 59 998, 20 09 566, 22 14 454, 22 15 191, 22 44 298, 23 13 331, 25 22 572, 31 37 808, 31 37 809, 31 51 343, 31 51 354, 31 51 355, 32 11 602, 32 35 017 and P 38 42 330 and are commercially available, for example under the trademarks Iriodin®, Timiron® and Xirona® from Merck KGaA, Darmstadt, Germany and/or Rona, USA. Particularly preferred pigment compositions comprise TiO₂/mica, Fe₂O₃/mica and/or TiO₂/Fe₂O₃/mica pigments. The pearlescent pigments may additionally have a layer of Berlin Blue or Carmine Red on the surface.

Preference is furthermore given to coated or uncoated BiOCl pigments, TiO₂- and/or Fe₂O₃-coated SiO₂, glass or Al₂O₃ flakes. The coating of the SiO₂ flakes with one or more metal oxides can be carried out, for example, as described in WO 93/08237 (wet-chemical coating) or DE-A 196 14 637 (CVD method).

The multilayered pigments disclosed, for example, in DE-A 196 18 563, DE-A 196 18 566, DE-A 196 18 569, DE-A 197 07 805, DE-A 197 07 806 and DE-A 197 46 067 are based on a flake-form, transparent, coloured or colourless matrix consisting of mica (synthetic or natural), SiO₂ flakes, glass flakes, Al₂O₃ flakes or polymer flakes and generally have a thickness of between 0.3 and 5 μm, in particular between 0.4 and 2.0 μm. The size in the other two dimensions is usually between 1 and 250 μm, preferably between 2 and 100 μm, and in particular between 5 and 40 μm. The multilayered pigments consist of the matrix (substrate) coated with metal oxides (at least 2). The coating of the substrate flakes mica, SiO₂ flakes, glass flakes or Al₂O₃ flakes with a plurality of layers is carried out in such a way that a layer structure preferably consisting of alternating high- and low-refractive-index layers is formed. The multilayered pigments preferably contain 2, 3, 4, 5, 6 or 7 layers, in particular 3, 4 or 5 layers. Suitable high-refractive-index metal oxides are, for example, titanium dioxide, zirconium oxide, zinc oxide, iron oxides, iron/titanium oxides (iron titanates) and/or chromium oxide, in particular TiO₂ and/or Fe₂O₃. The low-refractive-index oxides used are SiO₂ and Al₂O₃. However, it is also possible to employ MgF₂ or an organic polymer (for example acrylate) for this purpose. The coating of the substrate flakes can be carried out, for example, as described in WO 93/08237 (wet-chemical coating) or DE-A-196 14 637 (CVD process). Particularly preferred multilayered pigments based on mica (natural or synthetic), glass flakes, Al₂O₃ flakes, Fe₂O₃ flakes or SiO₂ flakes comprise a layer sequence TiO₂—SiO₂—TiO₂. The TiO₂ here can be in the rutile or anatase modification. It is preferably in the form of rutile.

The interference pigments are preferably pigments based on mica, glass flakes or SiO₂ flakes which are coated with coloured or colourless metal oxides, such as, for example, TiO₂, titanium suboxides, titanium oxynitrides, Fe₂O₃, Fe₃O₄, SnO₂, Cr₂O₃, ZnO, CuO, NiO, and other metal oxides, alone or in a mixture, in a single layer or in successive layers.

If the said pearlescent, interference and multilayered pigments comprise a TiO₂ layer, this can be in the rutile or anatase modification. It is preferably in the form of rutile. Rutilisation is known to the person skilled in the art and can be carried out, for example, as described in U.S. Pat. No. 4,038,099, U.S. Pat. No. 5,433,779, U.S. Pat. No. 6,626,989, WO 03/097749, U.S. Pat. No. 4,086,100, U.S. Pat. No. 4,867,794. Particular preference is given to rutilisation using tin oxide, as described, for example, in U.S. Pat. No. 4,867,794.

Suitable flake-form colorants are, in particular, pearlescent pigments, in particular based on mica, SiO₂ flakes or Al₂O₃ flakes, which are only covered with one metal-oxide layer, metal-effect pigments (Al flakes, bronzes), optically variable pigments (OVPs), liquid-crystal polymer pigments (LCPs) or holographic pigments.

The pigments of component B based on SiO₂ flakes are of course different from the effect pigment of component A.

The spherical colorants include, in particular, TiO₂, coloured SiO₂, CaSO₄, iron oxides, chromium oxides, carbon black, organic coloured pigments, such as, for example, anthraquinone pigments, quinacridone pigments, diketopyrrolopyrrole pigments, phthalocyanine pigments, azo pigments and isoindoline pigments. The needle-shaped pigments are preferably BiOCl, coloured glass fibres, α-FeOOH, organic coloured pigments, such as, for example, azo pigments, β-phthalocyanine CI Blue 15.3, Cromophtal Yellow 8GN (Ciba-Geigy), Irgalith Blue PD56 (Ciba-Geigy), azomethine copper complex CI Yellow 129 or Irgazine Yellow 5GT (Ciba-Geigy).

It is likewise possible to admix nanoscale dielectrics in order to improve the skin feel. Examples of admixtures of this type are Al₂O₃, SiO₂, ZnO or TiO₂, which are usually added in amounts of 0.01-15% of the formulation.

The pigment mixture according to the invention is simple and easy to handle. The pigment mixture can be incorporated into the application system by simple stirring-in. Components A and B can be added to the application system simultaneously, successively or as a mixture. Complex grinding and dispersion of the pigments is unnecessary.

The pigment mixture according to the invention can be used for the pigmenting of food colourings, for the finishing of foods, for example mass colouring or as a coating, in medicament coatings, for example in dragees and tablets, or in cosmetic formulations, such as lipsticks, lip gloss, eyeliner, eye shadow, rouge, sunscreen, pre-sun and after-sun compositions, make-ups, body lotions, bath gels, soaps, bath salts, toothpaste, hair gels, (volume) mascara, nail varnishes, compact powders, shampoos, loose powders and gels, etc.

The concentration of the pigment mixture in the application system to be pigmented is generally between 0.01 and 70% by weight, preferably between 0.1 and 50% by weight and in particular between 1.0 and 10% by weight, based on the total solids content of the system. It is generally dependent on the specific application and can be up to 100% in the case of loose powders. The use concentration of the pigment mixture according to the invention extends from 0.01% by weight in shampoo to 70% by weight in compact powders. In a mixture of red effect pigments with spherical fillers, for example SiO₂, the concentration can be 0.01-70% by weight in the formulation. The cosmetic products, such as, for example, nail varnishes, lipsticks, compact powders, shampoos, loose powders and gels, are distinguished by particularly interesting lustre effects.

The pigment mixture according to the invention can advantageously be employed in both decorative and care cosmetics. The use concentration and the mixing ratio of the red effect pigments with component B, in particular organic and inorganic coloured pigments and dyes, of natural or synthetic origin, such as, for example, chromium oxide, ultramarine, spherical SiO₂ or TiO₂ pigments, are dependent on the application medium and the effect to be achieved.

The red effect pigment can furthermore be mixed with commercially available fillers. Fillers which may be mentioned are, for example, natural and synthetic mica, glass beads or glass powder, nylon powder, pure or filled melamine resins, talc, glasses, kaolin, oxides or hydroxides of aluminium, magnesium, calcium or zinc, BiOCl, barium sulfate, calcium sulfate, calcium carbonate, magnesium carbonate, carbon, and physical or chemical combinations of these substances.

There are no restrictions regarding the particle shape of the filler. In accordance with requirements, it can be, for example, flake-form, spherical, needle-shaped, crystalline or amorphous.

The red effect pigments or the pigment mixture according to the invention can of course also be combined in the formulations with cosmetic raw materials and auxiliaries of any type. These include, inter alia, oils, fats, waxes, film formers, surfactants, antioxidants, such as, for example, vitamin C or vitamin E, stabilisers, odour intensifiers, silicone oils, emulsifiers, solvents, such as, for example, ethanol, or ethyl acetate or butyl acetate, preservatives and auxiliaries which generally determine applicational properties, such as, for example, thickeners and rheological additives, such as, for example, bentonites, hectorites, silicon dioxides, Ca silicates, gelatins, high-molecular-weight carbohydrates and/or surface-active auxiliaries, etc.

The formulations comprising the pigment mixtures according to the invention can belong to the lipophilic, hydrophilic or hydrophobic type. In the case of heterogeneous formulations having discrete aqueous and non-aqueous phases, the pigment mixtures according to the invention may in each case be present in only one of the two phases or alternatively distributed over both phases.

The pH of the formulations can be between 1 and 14, preferably between 2 and 11 and particularly preferably between 5 and 8.

No limits are set for the concentrations of the pigment mixtures according to the invention in the formulation. They can be—depending on the application—between 0.001 (rinse-off products, for example shower gels) and 100% (for example lustre-effect articles for particular applications).

The pigments according to the invention may furthermore also be combined with cosmetic active ingredients. Suitable active ingredients are, for example, insect repellents, inorganic UV filters, such as, for example, TiO₂, UV A/BC protective filters (for example OMC, B3 and MBC), also in encapsulated form, anti-ageing active ingredients, vitamins and derivatives thereof (for example vitamin A, C, E, etc.), self-tanning agents (for example DHA, erythrulose, inter alia), and further cosmetic active ingredients, such as, for example, bisabolol, LPO, VTA, ectoine, emblica, allantoin, bioflavonoids and derivatives thereof.

Organic UV filters are generally incorporated into cosmetic formulations in an amount of 0.5 to 10 percent by weight, preferably 1-8%, and inorganic filters in an amount of 0.1 to 30%.

The preparations according to the invention may in addition comprise further conventional skin-protecting or skin-care active ingredients. These may in principle be any active ingredients known to the person skilled in the art.

Particularly preferred active ingredients are pyrimidinecarboxylic acids and/or aryl oximes.

Of the cosmetic applications, particular mention should be made of the use of ectoine and ectoine derivatives for the care of aged, dry or irritated skin.

Thus, European patent application EP-A-0 671 161 describes, in particular, that ectoine and hydroxyectoine are employed in cosmetic preparations, such as powders, soaps, surfactant-containing cleansing products, lipsticks, rouge, make-up, care creams and sunscreen compositions.

Application forms of the cosmetic formulations which may be mentioned are, for example: solutions, suspensions, emulsions, PIT emulsions, pastes, ointments, gels, creams, lotions, powders, soaps, surfactant-containing cleansing compositions, oils, aerosols and sprays. Examples of other application forms are sticks, shampoos and shower preparations. Any desired customary excipients, auxiliaries and, if desired, further active ingredients may be added to the preparation.

Ointments, pastes, creams and gels may comprise the customary excipients, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary excipients, for example lactose, talc, silica, aluminium hydroxide, calcium silicate and polyamide powder, or mixtures of these substances Sprays may additionally comprise the customary propellants, for example chlorofluorocarbons, propane/butane or dimethyl ether.

Solutions and emulsions may comprise the customary excipients, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary excipients, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium meta-hydroxide, bentonite, agar-agar and tragacanth, or mixtures of these substances.

Soaps may comprise the customary excipients, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary excipients, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary excipients, such as synthetic oils, such as, for example, fatty acid esters, fatty alcohols, silicone oils, natural oils, such as vegetable oils and oily plant extracts, paraffin oils, lanolin oils, or mixtures of these substances.

The cosmetic preparations may exist in various forms. Thus, they can be, for example, a solution, a water-free preparation, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol, It is also advantageous to administer ectoines in encapsulated form, for example in collagen matrices and other conventional encapsulation materials, for example as cellulose encapsulations, in gelatin, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Further embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

Solid sticks consist of natural or synthetic waxes and oils, fatty alcohols, fatty acids, fatty acid esters, lanolin and other fatty substances.

If a preparation is formulated as an aerosol, the customary propellants, such as alkanes, fluoroalkanes and chlorofluoroalkanes, are generally used.

The cosmetic preparation may also be used to protect the hair against photochemical damage in order to prevent colour changes, bleaching or damage of a mechanical nature. In this case, a suitable formulation is in the form of a rinse-out shampoo, lotion, gel or emulsion, the preparation in question being applied before or after shampooing, before or after colouring or bleaching or before or after permanent waving. It is also possible to select a preparation in the form of a lotion or gel for styling or treating the hair, in the form of a lotion or gel for brushing or blow-waving, in the form of a hair lacquer, permanent waving composition, colorant or bleach for the hair. The preparation having light-protection properties may comprise adjuvants, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The invention thus furthermore also relates to formulations comprising the pigment according to Claim 1 according to the invention or the pigment mixture according to the invention in combination with at least one constituent selected from the group of absorbents, astringents, antimicrobial substances, antioxidants, antiperspirants, antifoaming agents, antistatics, binders, biological additives, bleaching agents, chelating agents, deodorants, emollients, emulsifiers, emulsion stabilisers, dyes, humectants, film formers, odour substances, flavour substances, preservatives, anticorrosion agents, cosmetic oils, solvents, oxidants, vegetable constituents, buffer substances, reducing agents, abrasives, surfactants, propellant gases, opacifiers, UV filters and UV absorbers, denaturing agents, viscosity regulators and vitamins.

The pharmaceutical and food products are coloured by adding the pigment mixture, preferably comprising the red effect pigment and colorants, such as, for example, natural or nature-identical dyes, in the desired mixing ratios to the product to be coloured in amounts of 0.005 to 15% by weight, preferably 0.01 to 100% by weight.

The admixing of natural or nature-identical dyes, organic or inorganic coloured pigments or colouring natural fruit and plant extracts which are approved for the foods sector enables the red colour effect of the effect pigments in the product to be influenced and at the same time enables novel iridescent colour effects to be achieved.

Suitable natural or nature-identical dyes are, in particular, E 101, E 104, E 110, E 124, E 131, E 132, E 140, E 141, E 151, E 160a. Furthermore, it is also possible to admix other coloured pigments with the flake-form effect pigments, such as, for example, E 171, E 172, E 153.

Besides the red effect pigments, the proportion of dyes, based on the food or pharmaceutical product, is preferably in the range from 0.5 to 25% by weight. The dyes employed can likewise be fruit and plant extracts, such as, for example, carrot juice, beetroot juice, elderberry juice, hibiscus juice, paprika extract, aronia extract.

The total concentration of all pigments in the product to be pigmented should not exceed 50% by weight, based on the product. It is generally dependent on the specific application.

Various active-ingredient additives, such as, for example, vitamins, enzymes, trace elements, proteins, carbohydrates, essential fats and/or minerals, can also be added to the food and pharmaceutical products, where the total amount of active ingredients, based on the food or pharmaceutical product, should not exceed 25% by weight. The amount of active ingredients or active-ingredient mixtures is preferably 0.01-20% by weight, based on the product.

The products are coloured by adding the effect pigment or effect-pigment mixture to the product to be coloured, alone or in combination with further pigments or colorants, directly or in the presence of water and/or an organic solvent in the desired mixing ratios, simultaneously or successively, during or after the manufacture thereof, before or after shaping (for example during extrusion, pelleting, use of an exponder, granulation, etc.). Admixing of the red effect pigments with pulverulent or loose powders is likewise possible.

The red effect pigments, alone or in the pigment mixture, can also be applied to the surface in order to colour the food and pharmaceutical products after shaping. In this case, the red effect pigment is generally mixed with an application medium and subsequently applied to the product using suitable application and spray devices. The application or coating composition then ensures the corresponding adhesion of the red pigments to the product surface. The latter is then coloured correspondingly.

On incorporation into the product matrix itself, the amount of red effect pigments used is preferably 0.5-40% by weight, in particular 1-30% by weight. In the case of surface colouring of food and pharmaceutical products, the amount used in the colouring or coating solution used is in the range 0.1-25% by weight, in particular 1-15% by weight. On use of the red effect pigments in pulverulent products, the use range is 0.05-50% by weight, in particular 2-10% by weight.

The coating solutions preferably comprise water or organic solvents, such as, for example, ethanol or isopropanol. The film former employed in the coating solutions is preferably a cellulose derivative, such as, for example, hydroxypropylmethylcellulose. Particular preference is given to application solutions comprising cellulose derivatives which, instead of water, comprise 5-80% by weight of a suitable organic solvent.

Compared with aqueous coating solutions, the alcoholic or alcoholic-aqueous, cellulose-containing application solutions have significant applicational advantages:
use of cooler drying air during the spray application
colouring of heat-sensitive products, such as, for example, vitamin-containing foods, with the red effect pigments is very readily possible.

Products which are suitable for colouring that may be mentioned are, in particular, coatings on all types of foods, in particular pigmented sugar and shellac coatings (alcoholic and aqueous), coatings with oils and waxes, with gum arabic and with cellulose grades (for example HPMC=hydroxypropylmethylcellulose), with starch and albumin derivatives, carrageenan and other substances known to the person skilled in the art which are suitable for coating. The red effect pigment or the pigment mixture is generally mixed with the application medium here and subsequently applied to the food or pharmaceutical product using suitable application and spray devices, or by hand. The application or coating composition then ensures the corresponding adhesion of the pigments to the food or pharmaceutical product surface. The latter is then coloured correspondingly. The application and coating solutions preferably comprise 0.1-20% by weight, in particular 2-15% by weight, of effect pigments.

Preferred dry powder mixtures for coatings comprise a cellulose derivative, such as, for example, hydroxypropylmethylcellulose, sodium carboxymethylcellulose, a release agent, such as, for example, lecithin or stearic acid, a gloss enhancer, such as, for example, maltodextrin and/or dextrose, and the red effect pigment. Dry powder mixtures of this type preferably comprise the red effect pigment in amounts of 0.01-50% by weight, in particular 0.5-40% by weight, based on the powder mixture. If necessary, dyes, flavourings, vitamins, sweeteners, etc., can be added to these dry powder mixtures.

Products which are suitable for colouring or coating are, for example, sugar products, cake decorations, compresses, dragees, chewing gum, gum products, fondant products, marzipan products, filling compositions, cocoa and fat glazes, chocolate and chocolate-containing products, ice cream, cereals, snack products, coating compositions, cake glazes, scattered sugar decorations, nonpareils, jelly and gelatin products, sweets, liquorice, icing, candyfloss, fat, sugar and cream compositions, blancmange, desserts, flan glaze, cold fruit soups, soft drinks and carbonated beverages, beverages with stabilising additives, such as, for example, carboxymethylcellulose, acidified and unacidified milk products, such as, for example, quark, yoghurt, cheese, cheese rinds, sausage casings, etc.

In the case of coated food and pharmaceutical products, it is possible to combine the red effect pigments with aroma substances (powder or liquid aromas), acids and/or with sweeteners, such as, for example, aspartame, in order additionally to reinforce the visual effect in terms of flavour.

The invention thus relates to all formulations from the foods and pharmaceuticals sector comprising the red effect pigments, alone or in combination with further pigments/pigment mixtures or dyes (natural or nature-identical), as colorants.

A further major area of application is in the pharmaceutical and OTC sector for colouring or as a coating for tablets, gelatin capsules, dragees, ointments, cough mixture, etc. In combination with conventional coatings, such as polymethacrylates and cellulose grades, for example HPMC, the red effect pigment can be employed in a variety of ways for colouring and finishing the products.

The invention thus also relates to formulations comprising the pigment mixture according to the invention.

The following examples are intended to explain the invention, but without limiting it.

### EXAMPLES

### Example 1

100 g of SiO₂ flakes having a thickness of 365 nm are heated to 75° C. in 2 l of deionised water. 1137 ml of FeCl₃ solution (corresponding to 132% of Fe₂O₃) are added with stirring. The pH of the reaction mixture is kept constant at 3 by addition of sodium hydroxide solution (30%). After addition of the FeCl₃ solution, the pH is raised to pH 5 using sodium hydroxide solution (30%). The product is filtered off and rinsed with deionised water. After drying at 110° C., the product is calcined at 800° C.

The pigment has the following PDS (particle distribution size) values (measured using a Malvern Mastersizer 2000):

### D₁₀=9.21

### D₅₀=19.5

### D₉₀=37.2

The red effect pigment exhibits the following L,a,b values (measured using an Eta measuring instrument, measurement angles 50°/140°):

### L=135.1

a=102.6
b=53.3

### USE EXAMPLES

### Example A1

### Eye Shadow Gel

Phase A

| Raw material | Source of supply | INCI | [%] |
|---|---|---|---|
| Red pigment | Merck KGaA/ | CI 77491 (IRON | 15.00 |
| according | Rona ® | OXIDES), SILICA | |
| to Example 1 | | | |
| Micronasphere ® | Merck KGaA/ | MICA, SILICA | 8.00 |
| M | Rona ® | | |
| Carbopol | Noveon | ACRYLATES/C10-30 | 0.40 |
| Ultrez 21 | | ALKYL ACRYLATE | |
| | | CROSSPOLYMER | |
| Citric acid | Merck KGaA/ | CITRIC ACID | 0.00 |
| monohydrate | Rona ® | | |
| Water | | AQUA (WATER) | to 100 |

Phase B

| Raw material | Source of supply | INCI | [%] |
|---|---|---|---|
| Glycerin | Merck KGaA/ | GLYCERIN | 3.00 |
| | Rona ® | | |
| Preservatives | | | q.s. |
| Triethanolamine | | TRIETHANOLAMINE | 0.70 |
| Water | | AQUA (WATER) | 13.00 |

Phase C

| Raw material | Source of supply | INCI | [%] |
|---|---|---|---|
| Lubrajel DV | | PROPYLENE GLYCOL, | 5.00 |
| | | POLYGLYCERYL | |
| | | METHACRYLATE | |

### Preparation:

Disperse red pigment and Micronasphere® in the water of phase A. Acidify using a few drops of citric acid in order to lower the viscosity, scatter in Carbopol with stirring. After complete dissolution, slowly stir in the pre-dissolved phase B and subsequently phase C. Finally, adjust the pH to between 7.0 and 7.5.

### Example A2

### Powder Eye Shadow

Phase A

| Raw material | Source of supply | INCI | [%] |
|---|---|---|---|
| Red pigment | Merck KGaA/Rona ® | CI 77491 (IRON | 30.00 |
| according | | OXIDES), SILICA | |
| to Example 1 | | | |
| Talc | Merck KGaA/Rona ® | TALC | 49.50 |
| Magnesium stearate | Merck KGaA/Rona ® | MAGNESIUM | 2.50 |
| | | STEARATE | |
| Potato starch | Suedstaerke GmbH | SOLANUM | 7.50 |
| | | TUBEROSUM | |
| | | (POTATO | |
| | | STARCH) | |

Phase B

| Raw material | Source of supply | INCI | [%] |
|---|---|---|---|
| Isopropyl | Cognis GmbH | ISOPROPYL | 9.34 |
| stearate | | STEARATE | |
| Cetyl | Merck KGaA/Rona ® | CETYL | 0.53 |
| palmitate | | PALMITATE | |
| Ewalin 1751 | H. Erhard Wagner | PETROLATUM | 0.53 |
| | GmbH | | |
| Propyl 4- | Merck KGaA/Rona ® | PROPYLPARABEN | 0.10 |
| hydroxy- | | | |
| benzoate | | | |

### Preparation:

Combine and pre-mix the constituents of phase A. Subsequently add the molten phase B dropwise to the powder mixture with stirring. The powders are pressed at 40-50 bar.

### Suntest:

In order to assess the light stability, half of the compact powder is covered during the exposure. The powders are then introduced into the Suntest (manufacturer: Hereaus Suntest CPS, 72 W/m² xenon lamp) for 8 hours. After exposure for 8 hours, no discoloration of the powder in the Suntest is observed. The red pigment is absolutely light-stable.

### Example A3

### Lipstick

Phase A

| Raw material | Source of supply | INCI | [%] |
|---|---|---|---|
| Red pigment | Merck KGaA/Rona ® | CI 77491 (IRON | 12.00 |
| according | | OXIDES), SILICA | |
| to Example 1 | | | |
| Ronastar ® | Merck KGaA/Rona ® | CALCIUM ALUMINUM | 3.00 |
| Purple | | BOROSILICATE, | |
| Sparks | | CI77891 (TITANIUM | |
| | | DIOXIDE), SILICA, | |
| | | TIN OXIDE | |

Phase B

| Raw material | Source of supply | INCI | [%] |
|---|---|---|---|
| Beeswax | Merck KGaA/Rona ® | Cera Alba | 8.75 |
| | | (Beeswax) | |
| Paracera | Paramelt | COPERNICIA | 5.25 |
| C44 | | CERIFERA | |
| | | (CARNAUBA WAX), | |
| | | CERESIN | |
| Adeps | Henry Lamotte | LANOLIN | 3.50 |
| Lanae | GmbH | | |
| Isopropyl | Cognis GmbH | Isopropyl | 5.60 |
| myristate | | Myristate | |
| Liquid | Merck KGaA/Rona ® | PARAFFINUM | 2.10 |
| paraffin | | LIQUIDUM (MINERAL | |
| | | OIL) | |
| Castor oil | Henry Lamotte | RICINUS COMMUNIS | 59.65 |
| | GmbH | (CASTOR OIL) | |
| Oxynex K | Merck KGaA/Rona ® | PEG-8, | 0.05 |
| liquid | | TOCOPHEROL, | |
| | | ASCORBYL | |
| | | PALMITATE | |
| | | ASCORBIC ACID, | |
| | | CITRIC ACID | |
| Propyl 4- | Merck KGaA/Rona ® | PROPYLPARABEN | 0.10 |
| hydroxy- | | | |
| benzoate | | | |

### Preparation:

The constituents of phase B are heated to 75° C. and melted. The pigments of phase A are added, and everything is stirred well, The lipstick material is then stirred for 15 minutes in the casting apparatus heated to 65° C. The homogeneous melt is poured into the casting mould which is pre-warmed to 55° C. The moulds are subsequently cooled, and the cold castings are removed. After warming of the lipsticks to room temperature, the lipsticks are briefly flame-treated.

### Example A4

### Nail Varnish

| Raw material | Source of supply | INCI | [%] |
|---|---|---|---|
| Red pigment | Merck KGaA/ | CI 77491 (IRON | 2.00 |
| according | Rona ® | OXIDES), SILICA | |
| to Example 1 | | | |
| Nailsyn ® | Merck KGaA/ | CI 77163 (Bismuth | 1.00 |
| Sterling 60 | Rona ® | OXYCHLORIDE), | |
| Silver | | BUTYL ACETATE, | |
| | | NITROCELLULOSE, | |
| | | ISOPROPYL | |
| | | ALCOHOL, ETHYL | |
| | | ACETATE | |
| | | STEARNALKONIUM | |
| | | HECTORITE | |
| Thixotropic | Durlin/ | BUTYL ACETATE, | 97.00 |
| nail varnish | Bergerac NC | ETHYL ACETATE | |
| base 155 | | NITROCELLULOSE, | |
| | | ACETYL TRIBUTYL | |
| | | CITRATE, PHTHALIC | |
| | | ANHYDRIDE/TRIMELLITIC | |
| | | ANHYDRIDE/GLYCOLS | |
| | | COPOLYMER, | |
| | | ISOPROPYL | |
| | | ALCOHOL, | |
| | | STEARALKONIUM | |
| | | HECTORITE, ADIPIC | |
| | | ACID/FUMARIC | |
| | | ACID/PHTHALIC | |
| | | ACID/TRICYCLODECANE | |
| | | DIMETHANOL | |
| | | COPOLYMER, CITRIC | |
| | | ACID | |

### Preparation:

The pigment and Nailsyn® Sterling 60 Silver are weighed out together with the varnish base, mixed well by hand using a spatula and subsequently stirred at 1000 rpm for 10 min.

### Example A5

### Volume Mascara

Phase A

| Raw material | Source of supply | INCI | [%] |
|---|---|---|---|
| Red pigment | Merck KGaA/Rona ® | CI 77491 (IRON | 10.00 |
| according | | OXIDES), SILICA | |
| to Example 1 | | | |
| Satin mica | Merck KGaA/Rona ® | MICA | 2.00 |

Phase B

| Raw material | Source of supply | INCI | [%] |
|---|---|---|---|
| Dow Corning | Dow Corning | PHENYL | 2.00 |
| 556 | | TRIMETHICONE | |
| Tegosoft CT | Degussa- | CAPRYLIC CAPRIC | 2.50 |
| | Goldschmidt AG | TRIGLYCERIDE | |
| Syncrowachs | Croda GmbH | TRIBEHENIN | 3.50 |
| HRC | | | |
| Tegin M | Degussa- | GLYCERYL | 3.50 |
| | Goldschmidt AG | STEARATE | |
| Beeswax | Merck KGaA/Rona ® | CERA ALBA | 3.00 |
| | | (BEESWAX) | |
| Stearic | Merck KGaA/Rona ® | STEARIC ACID | 5.00 |
| acid | | | |
| Phenonip | Nipa Laboratorien | PHENOXYETHANOL, | 0.80 |
| | GmbH | BUTYLPARABEN, | |
| | | ETHYLPARABEN, | |
| | | PROPYLPARABEN, | |
| | | METHYLPARABEN | |
| RonaCare ® | Merck KGaA/Rona ® | TOCOPHEROL | 0.50 |
| tocopherol | | ACETATE | |
| acetate | | | |
| Dermacryl | Amerchol | ACRYLATES/OCTYL- | 3.50 |
| 79 | | ACRYLAMIDE | |
| | | COPOLYMER | |

Phase C

| Raw material | Source of supply | INCI | [%] |
|---|---|---|---|
| Water | | AQUA (WATER) | 59.15 |
| (demineralised) | | | |
| AMP Ultra | Angus Chemie | AMINOMETHYL | 1.25 |
| PC 1000 | GmbH | PROPANOL | |
| 1,3-Butanediol | Merck KGaA/Rona ® | BUTYLENE GLYCOL | 1.00 |
| RonaCare ® | Merck KGaA/Rona ® | UREA, DISODIUM | 0.50 |
| Biotin Plus | | PHOSPHATE, | |
| | | BIOTIN, CITRIC ACID | |

Phase D

| Raw material | Source of supply | INCI | [%] |
|---|---|---|---|
| Germall 115 | ISP Global | IMIDAZOLIDINYL | 0.30 |
| | Technologies | UREA | |
| Water | | AQUA (WATER) | 1.50 |
| (demineralised) | | | |

### Preparation:

Melt all constituents of phase B apart from Dermacryl 79 together at about 85° C., add Dermacryl 79 with stirring and stir for 20 min until everything is homogeneously dispersed. Heat the constituents of phase C to about 85° C. Stir the red pigment of phase A into phase C. Add phase C to phase B, continue stirring and homogenise at 8000 rpm for 1 min using an Ultra-Turrax T25. Allow to cool with stirring, and add phase D at 40° C.

### Example A6

### Soap

| Raw material | Source of supply | INCI | [%] |
|---|---|---|---|
| Red pigment | Merck KGaA/ | CI 77491 (IRON | 1.50 |
| according | Rona ® | OXIDES), SILICA | |
| to Example 1 | | | |
| Ronastar ® | Merck KGaA/ | CALCIUM ALUMINUM | 0.50 |
| Noble Sparks | Rona ® | BOROSILICATE, | |
| | | SILICA, CI 77891 | |
| | | (TITANIUM DIOXIDE), | |
| | | TIN OXIDE | |
| Transparent | Jean Charles | SODIUM PALMATE, | 98.00 |
| soap base | (USA) | SODIUM LAURETH | |
| | | SULFATE, SODIUM | |
| | | STEARATE, SODIUM | |
| | | MYRISTATE, SODIUM | |
| | | COCOYL | |
| | | ISETHIONATE, | |
| | | TRIETHANOLAMINE, | |
| | | AQUA | |
| | | (WATER), GLYCERIN, | |
| | | SORBITOL, | |
| | | PROPYLENE | |
| | | GLYCOL, | |
| | | FRAGRANCE | |

### Preparation:

All constituents are mixed homogeneously.

### Example A7

### Preparation of Hard Caramels

| Raw material | % | Sources of supply: |
|---|---|---|
| Sugar | 41% | Südzucker |
| Water | 17.118% | |
| Glucose syrup | 41% C* sweet | Cerestar, Krefeld |
| Red pigment accord- | 0.082% (0.1% based on | Merck KGaA, |
| ing to Example 1 | the casting composition) | Darmstadt |
| E 104 1:100 dil. | 0.4% Sikovit | BASF, Ludwigshafen |
| Aroma | 0.4% | Dragaco, Holzminden |
| | (banana 9/030388) | |

The sugar is heated to 100° C. with the water, and glucose syrup is then added. The solution is subsequently heated to 145° C. After addition of the red effect pigment, the colour solution and the aroma, the caramel solution is poured into greased moulds using a pouring funnel. Finally, the mixture is allowed to cool for two hours. The red pigment can either be mixed with the sugar or added as a mixture with the glucose syrup. This variant contains no acid since this would make the caramelisation excessive.

### Example A8

### Production of Gelatin Articles

| Raw material | % | Sources of supply: |
|---|---|---|
| Water | 10.48% | |
| Sugar | 31.45% | Südzucker |
| Glucose syrup | 31.45% C*sweet | Cerestar, Krefeld |
| Red pigment according | 0.38% (0.4% based on | Merck KGaA, |
| to Example 1 | the casting composition) | Darmstadt |
| Citric acid 1:1 dil. | 2.51% | Merck KGaA, |
| | | Darmstadt |
| Gelatin | 7.86% 260 Bloom | DGF, Eberbach |
| Water | 15.748% | |
| Aroma | 0.122% (blackcurrant | Dragoco, Holzminden |
| | 9/695750) | |

Firstly, the gelatin is softened at 60° C. with twice the amount of water. Sugar and water are heated to 100° C., and the glucose syrup is then added. The mixture is heated further to 120° C. and then allowed to cool to about 85° C. The red effect pigment, the citric acid, the aroma and the gelatin solution are stirred in, and the de-aerated gelatin mixture is transferred into greased moulds using the pouring funnel. The product is allowed to cool for about 16 hours.

### Further Embodiments

The red effect pigment can again be mixed directly with the sugar here or introduced with the glucose syrup.
Instead of pouring into moulds, the traditional technique with negative moulds in mould powder for the production of gelatin articles can also be used here.

### Example A9

### Coating of Tablets

a) Initial weight 1 kg of white tablets d=8 mm, G=200 mg

Solution for film coating:

| | | |
|---|---|---|
| 6% | Sepifilm Lp10 | Seppic |
| | (mixture of hydroxypropylmethyl- | |
| | cellulose, stearic acid and | |
| | microcrystalline cellulose) | |
| 5% | Red pigment according to | Merck KGaA, Darmstadt |
| | Example 1 | |
| 89% | Water | |

| | |
|---|---|
| Total application amount: | 200 g |

This corresponds to 1.2 mg of polymer/cm² of tablet surface

### Preparation of the Film-Coating Solution:

The red effect pigment is stirred into water. Additional dyes are subsequently added if desired. Finally, the film former (HPMC) is scattered into the suspension. Due to the increasing viscosity, the stirring speed must also be increased correspondingly. After about 40-60 minutes, the HPMC has completely dissolved and the solution can then be sprayed onto the tablets.
The spray application is carried out by means of a common standard coating method.

1. Effect pigments based on transparent, undoped SiO₂ flakes, characterised in that they are covered with an iron-oxide layer having a thickness of 30-150 nm, all SiO₂ flakes have an identical thickness, and the thickness of the SiO₂ flakes is 250-400 nm.

2. Effect pigments according to claim 1, characterised in that the iron oxide is Fe₂O₃.

3. Process for the preparation of the effect pigments according to claim 1, characterised in that the SiO₂ flakes are suspended in water and coated completely with iron oxide by addition and precipitation of corresponding inorganic iron compounds, where the pH necessary for the precipitation of iron oxide is set and kept constant by simultaneous addition of acid or base, and the coated substrate is subsequently separated off from the aqueous suspension, dried and optionally calcined, and where the layer thicknesses of the individual layers are set in such a way that the thickness of the pigment after drying and optionally calcination is not greater than 500 (±30) nm.

4. Process for the preparation of the effect pigments according to claim 1, characterised in that the SiO₂ flakes are covered with iron oxide by means of CVD coating in such a way that no open edges remain, where the layer thicknesses of the individual layers are set in such a way that the thickness of the pigment after drying and optional calcination is not greater than 500 (±30) nm.

5. Pigment mixture consisting of at least two components A and B, characterised in that component A is the effect pigment according to claim 1 and component B comprises inorganic pigments, organic pigments, dyes and/or fillers which consist of flake-form, needle-shaped, spherical or irregularly shaped particles.

6. Pigment mixture according to claim 5, characterised in that the colorant of component B is a pearlescent pigment, a multilayered pigment and/or an interference pigment or a mixture thereof.

7. Pigment mixture according to claim 5, characterised in that component A and component B are mixed in the weight ratio 99:1 to 50:50.

8. A formulation of claim 11 which is a cosmetic formulation and in the foods and pharmaceuticals sector.

9. A formulation of claim 11 which is a pharmaceutical product, characterised in that the effect pigment is employed alone or in combination with further pigments and/or colorants in combination with aroma substances and/or sweeteners.

10. A formulation of claim 11 which is a food or pharmaceutical product, characterised in that the pigment mixture is employed alone or in combination with further pigments and/or colorants in combination with aroma substances and/or sweeteners.

11. Formulations comprising a pigment mixture according to claim 5.

12. Formulations according to claim 12 comprising the pigment mixture and at least one constituent selected from the group of absorbents, astringents, antimicrobial substances, antioxidants, antiperspirants, antifoaming agents, antistatics, binders, biological additives, bleaching agents, chelating agents, deodorants, emollients, emulsifiers, emulsion stabilisers, dyes, humectants, film formers, odour substances, flavour substances, preservatives, anticorrosion agents, cosmetic oils, solvents, oxidants, vegetable constituents, buffer substances, reducing agents, abrasives, surfactants, propellant gases, opacifiers, UV filters and UV absorbers, denaturing agents, viscosity regulators and vitamins.

13. Process for the preparation of a formulation characterised in that components A and B of the pigment mixture according to claim 5 are added to an application system simultaneously, successively or as a mixture.
